(19) United States
(12) Patent Application Publication
(10) Pub. No.: US 2003/0236223 A1
(43) Pub. Date: Dec. 25, 2003

(54) Metastasis modulating activity of highly sulfated oligosaccharides

(75) Inventors: Thomas E. Wagner, Greer, SC (US); Xianzhang Yu, Mauldin, SC (US)

Correspondence Address: FOLEY AND LARDNER, SUITE 500, 3000 K STREET NW, WASHINGTON, DC, 20007, US

(73) Assignee: Greenville Hospital System

(21) Appl. No.: 10/409,607

(22) Filed: Apr. 9, 2003

Related U.S. Application Data
(60) Provisional application No. 60/370,730, filed on Apr. 9, 2002.

(51) Int. Cl. A61K 31/724

(52) U.S. Cl. 514/58

(57) ABSTRACT

The present invention provides compositions and methods for slowing tumor growth, decreasing cell proliferation, decreasing neovascularization and blocking metastatic spread using highly sulfated oligosaccharides. The compositions described herein are useful for cancer therapy and may be optionally complexed to at least one therapeutic agent.

### FIELD OF THE INVENTION

The present invention relates generally to compositions and methods of using sulfated oligosaccharides for slowing tumor growth and treating cancer.

### BACKGROUND OF THE INVENTION

Heparin, a mucopolysaccharide, is a constituent of various tissues, especially liver and lung, and mast cells in several mammalian species. Chemically, it has been described as an α, β glycosidically linked sulfated copolymer of D-glucosamine and D-glucuronic acid. It is polydisperse, with a molecular weight range from about 5,000 to 40,000. Within a given chain, there are also structural variations such as the varying degrees of sulfation, N-acetylation and C-5 epimerization in the uronic acid residue.

Heparins, in combination with certain steroids, can inhibit the growth of responsive tumors when administered in the proper dose range and with proper ratio of steroid, and even promote regression at somewhat higher doses and ratios. A major disadvantage of this treatment is that heparins can cause resumption of rapid tumor growth when administered at even higher dose levels and ratios to steroid. The apparent presence of both positive and negative regulators of tumor growth in heparin may create problems in properly administering the drug. Another disadvantage of heparin derives from the anticoagulant activity of heparin, restricting its use to low dosage levels or to oral administration in order to avoid bleeding.

Cyclodextrins (CDs) are saccharide compounds containing at least six glucopyranose units forming a ring or toroid shaped molecule, which therefore has no end groups. Although cyclodextrins with up to twelve glucopyranose units are known, only the first three homologs have been studied extensively. These compounds have a simple, well-defined chemical structure. The initial discovery of the cyclodextrins as degradation products of starch was made at about the turn of the century, and Schardinger showed that these compounds could be prepared by the action of Bacillus macerans amylase upon starch. In older literature, the compounds are often referred to as Schardinger dextrins. They are also sometimes called cycloamyloses.

The present inventors have determined that sulfated oligosaccharides, including sulfated cyclodextrins, can be used to slow tumor growth. These sulfated compounds typically do not have the negative effects normally associated with heparin treatment. Moreover, sulfated cyclodextrins have a cavity and therefore can carry therapeutic agents to a particular site and enhance its therapeutic effect in cancer treatment.

### SUMMARY OF THE INVENTION

In one aspect of the present invention is a pharmaceutical composition comprising a highly sulfated oligosaccharide and a pharmaceutically suitable excipient. Preferably, the highly sulfated oligosaccharide is a sulfated cyclodextrin and still preferred, the sulfated cyclodextrin is β-cyclodextrin tetradecasulfate (β-CD-TDS).

Also contemplated in the instant invention is a method for slowing tumor growth, decreasing cell proliferation, decreasing neovascularization and blocking metastatic spread comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient. Preferably, the highly sulfated oligosaccharide is a sulfated cyclodextrin and still preferred, the sulfated cyclodextrin is β-cyclodextrin tetradecasulfate. The sulfated cyclodextrin is preferably complexed to at least one therapeutic agent. Preferred therapeutic agents include antitumor drugs, antineoplastic drugs, cytokines, and anti-angiogenesis agents.

The instant invention also provides for a kit for slowing tumor growth, decreasing cell proliferation, decreasing neovascularization and blocking metastatic spread comprising (i) a first container that comprises a highly sulfated oligosaccharide and (ii) instructions for use. Preferably, the highly sulfated oligosaccharide is a sulfated cyclodextrin and still preferred, the sulfated cyclodextrin is β-cyclodextrin tetradecasulfate. The sulfated cyclodextrin is preferably complexed to at least one therapeutic agent.

### BRIEF DESCRIPTION OF THE DRAWINGS

FIG. 1 is a graph of B16 cell proliferation as a function of increasing β-cyclodextrin tetradecasulfate concentration.

FIG. 2 is a digital picture of lung tissue from mice treated (A) with and (B) without β-cyclodextrin tetradecasulfate.

### DETAILED DESCRIPTION OF THE PREFERRED EMBODIMENTS

It has been discovered by the present inventors that a sulfated oligosaccharide effectively slows tumor growth and blocks metastatic spread without exhibiting the undesirable properties of heparin. Additionally, applicants have found that a cyclodextrin derivative, namely a cyclodextrin sulfate, is effective when administered orally.

Pharmaceutical Composition

Highly Sulfated Oligosaccharide

The present invention contemplates a pharmaceutical composition that comprises a highly sulfated oligosaccharide and a pharmaceutically suitable excipient. A highly sulfated oligosaccharide ideally has at least one sulfur group to one saccharide unit. Preferably, the highly sulfated oligosaccharide has at least two sulfur groups per saaccharide unit, although one sulfur group per two glucopyranose units is also contemplated. In a preferred embodiment, the highly sulfated oligosaccharide is a cyclodextrin sulfate. As described herein, a cyclodextrin sulfate is a cyclodextrin derivative. It is contemplated in the present invention that any cyclodextrin, cyclodextrin derivative, analog, isomer, or combination of cyclodextrin molecules that is/are sulfated is a suitable oligosaccharide for use in the present invention.

Cyclodextrins are chiral, toroidal-shaped molecules formed by the action of the enzyme cyclodextrin transglycosylase on starch. These cyclic oligomers contain from 6 to 12 glucose units bonded through α-(1,4)-linkages. The three smallest homologs, α-cyclodextrin, β-cyclodextrin and γ-cyclodextrin are available commercially; larger homologs must be produced and isolated individually. The common designations of the lower molecular weight α-, β- and γ-CDs are used throughout this specification, wherein the number of glucopyranose units is 6, 7, or 8, respectively.

Topographically, the CDs may be represented as a torus, the upper rim of which is lined with primary —CH₂ OH groups, and the lower rim with secondary hydroxyl groups. Coaxially aligned with the torus is a channel-like cavity of about 5-8 Angstroms in diameter for the α-, β, and γ-CDs. These cavities make the cyclodextrins capable of forming inclusion compounds with hydrophobic guest molecules of suitable diameters (i.e., molecules which fit entirely or at least partially into the cyclodextrin cavity). Therefore, a particularly important use for cyclodextrins is as a carrier for drug molecules and antigens, which may be entrapped in the internal cavity thereof. The sulfated cyclodextrin of the present invention is preferably complexed to a therapeutic agent.

Various chemical modifications can first be made to the cyclodextrin molecule before providing a sulfated cyclodextrin. For example, at least one of the free hydroxyl groups on carbons 2, 3, or 6 can be replaced with an amino group. This modified cyclodextrin can then be sulfated through a sulfonamide linkage (i.e., —NH—SO₃). Such CD derivatives are well known in the art.

A reasonably large number of other CD derivatives have been prepared and described in the literature. In general, these chemically modified CDs are formed by reaction of the primary or secondary hydroxyl groups attached to carbons 2, 3 or 6, without disturbing the α (1→4) hemiacetal linkages. A review of such preparations is given in “Tetrahedron Report Number 147, Synthesis of Chemically Modified Cyclodextrins”, A. P. Croft and R. A. Bartsch, Tetrahedron 39(9):1417-1474 (1983). In particular, α-, β-, and γ-CD sulfates (Na salt) are shown as Compound Nos. 207, 208 and 209 in Tetrahedron Report No. 147 (supra) Table 26, p. 1456. The cyclodextrin derivatives described in U.S. Pat. No. 6,165,995 are also suitable for the present invention, so long as they are sulfated.

A preferred embodiment of the present invention is a sulfated β-cyclodextrin. Still preferred, the sulfated β-cylcodextrin is a β-cyclodextrin tetradecasulfate. As discussed supra, various degrees of sulfation per glucopyranose unit can be employed. An average of one sulfate group per two glucose units or one sulfate groups per glucopyranose unit are preferred. Especially preferred is β-CD-TDS which has an average of two sulfate groups per glucopyranose unit.

Methods

The pharmaceutical compositions of the present invention are useful for slowing tumor growth, decreasing cell proliferation and neovascularization, and blocking metastatic spread.

Method for Slowing Tumor Growth

Described herein is a method for slowing tumor growth, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient. In a preferred embodiment, the sulfated oligosaccharide is a sulfated cyclodextrin. Still preferred, sulfated cyclodextrin is a α, β, or γ cyclodextrin. For example, the sulfated cyclodextrin may be β-cyclodextrin tetradecasulfate

In another aspect of the present invention, the sulfated cyclodextrin is complexed to a therapeutic agent. As discussed below, therapeutic agents suitable for the therapeutic compositions described herein include those that are hydrophobic and have a molecular structure smaller than the cavity of the sulfated cyclodextrin.

Method for Decreasing Cell Proliferation

In a related vein, a method for decreasing cell proliferation, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient, is also described. Preferably, the highly sulfated oligosaccharide is a sulfated cyclodextrin and the cell is a melanoma cell. More preferably, the sulfated cyclodextrin is a α, β, or γ cyclodextrin. Still preferred, the sulfated cyclodextrin is β-cyclodextrin tetradecasulfate. In another embodiment of the present invention, the sulfated cyclodextrin is complexed to a therapeutic agent.

Method for Decreasing Neovascularization

Similarly, a method for decreasing neovascularization, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient is described herein. In a preferred embodiment, the highly sulfated oligosaccharide is a sulfated cyclodextrin such as a β-cyclodextrin tetradecasulfate. Also preferred, the sulfated cyclodextrin is a α, β, or γ cyclodextrin. Still preferred, the sulfated cyclodextrin is complexed to a therapeutic agent.

Method for Blocking Metastatic Spread

Contemplated herein is also a method for blocking metastatic spread, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient. Preferably, the highly sulfated oligosaccharide is a sulfated cyclodextrin. Still preferred, the sulfated cyclodextrin is an α, β, or γ cyclodextrin. Also preferred, the sulfated cyclodextrin is β-cyclodextrin tetradecasulfate and is complexed with a therapeutic agent.

Therapeutic Agents

The pharmaceutical compositions of the present invention are preferably complexed to a therapeutic agent. Therapeutic agents of the present invention include antitumor drugs, antineoplastic drugs, cytokines, and anti-angiogenesis agents. Conventional anti-tumor agents suitable for the present invention include adriamycin, cisplatin, colchicine, CCNU (Lomastine), BCNU (Carmustine), Actinomycin D, 5-fluorouracil, thiotepa, cytosinearabinoside, cyclophosphamide, mitomycin C, and the like. Other preferred therapeutic agents include, but are not limited to thalidomide, deoxyuridine, pyridine, mercaptopurines, toxins such as aflatoxins, ganciclovir, furosemide, indomethacin, chlorpromazine, methotrexate, cevine derivatives and analogs including cevadines, desatrines, veratridine, and various purine and pyrimidine derivatives and analogs including 5′-fluoro-2′-deoxyuridine, and allopurinol. As long as the therapeutic agent is hydrophobic in character and has a molecular structure smaller than the cavity of the sulfated cyclodextrin, it is suitable for complexing with the pharmaceutical compositions of the instant invention.

Pharmaceutically Suitable Excipients

In yet another aspect of the present invention is a composition comprising a highly sulfated oligosaccharide and a pharmaceutically suitable excipient in which the sulfated oligosaccharide is suspended. Such a pharmaceutical composition consists of the oligosaccharide in a form suitable for administration to a subject, or can comprise more than one pharmaceutically suitable excipient, one or more additional ingredient, or some combination of these.

For oral administration, the pharmaceutical compositions can take the form of, for example, tablets or capsules prepared by conventional means with pharmaceutically acceptable excipients such as binding agents (e.g., gelatin, acacia, pregelatinized maize starch, polyvinylpyrrolidone and hydroxypropyl methylcellulose); fillers (calcium carbonate, sodium carbonate, lactose, microcrystalline cellulose, calcium phosphate, calcium hydrogen phosphate and sodium phosphate); lubricants (e.g., magnesium stearate, stearic acid, silica and talc); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. In a preferred embodiment, the pharmaceutical composition of the present invention is given orally. The dosage range is preferably 0.1-2.0 mg/kg body weight per day.

Preparations for oral administration can be suitably formulated to give controlled release of the active compound.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The pharmaceutical composition of the present invention can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

Douche preparations or suspensions for vaginal irrigation can be made by combining the composition described herein, with a pharmaceutically acceptable liquid carrier. As is known in the art, douche preparations can be administered using, and can be packaged within, a delivery device adapted to the vaginal anatomy of the subject. Douche preparations can further comprise various additional ingredients, including antioxidants, antibiotics, antifungal agents, and preservatives.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include humans and other mammals

The invention is now described with reference to the following examples, which are provided for illustration only. The invention is not limited to the examples, but rather includes all variations which are evident as a result of the teaching provided therein. These examples demonstrate that, a highly sulfated oligosaccharide can impact tumor growth and metastasis.

### EXAMPLES

### Example 1

B16 Murine Melanoma Cell Proliferation Following β-cyclodextrin Tetradecasulfate Treatment

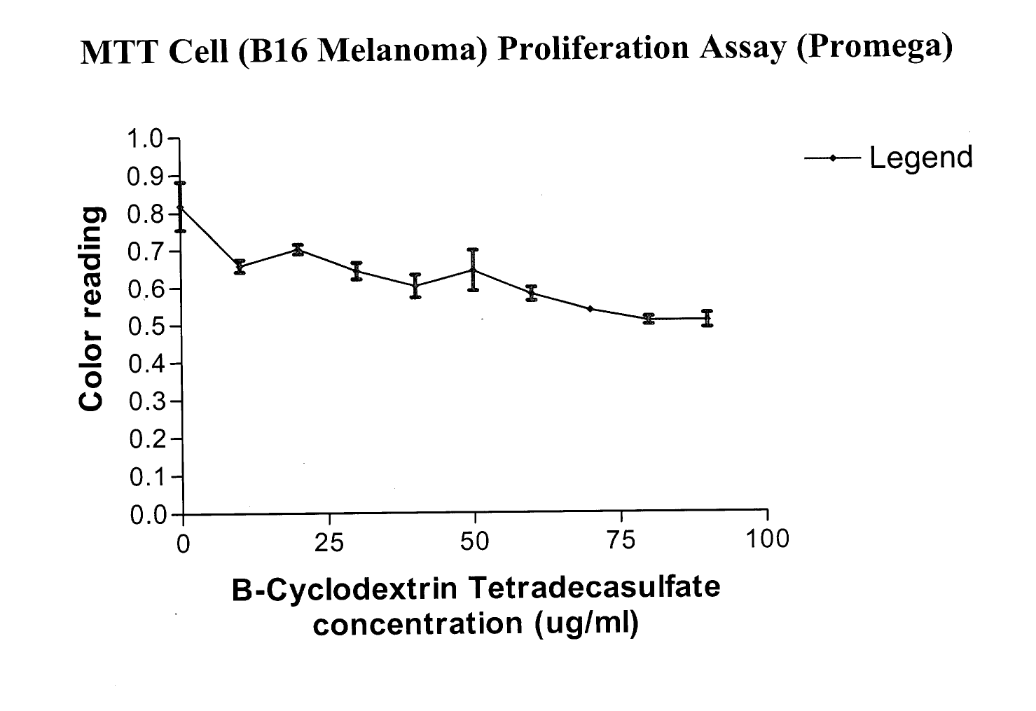

5000 cells/well of murine B16 melanoma cells were plated in a 96-well plate and incubated for 48 hours with varying concentrations of cyclodextrin tetradecasulfate (0-90 micrograms/ml). Non-radioactive assay solution was prepared according to manufacturer's protocol (Promega Cell Titer 96 AQᵤₑₒᵤₛ Assay Reagents). Cells were read using a Biorad benchmark microplate reader 2 hours after the assay solution was added to the well. FIG. 1 indicates that as cell concentration of β-cyclodextrin tetradecasulfate concentration increases, cell proliferation decreases.

### Example 2

Lung Metastasis in Tumorigenic Mice Treated with β-cyclodextrin Tetradecasulfate

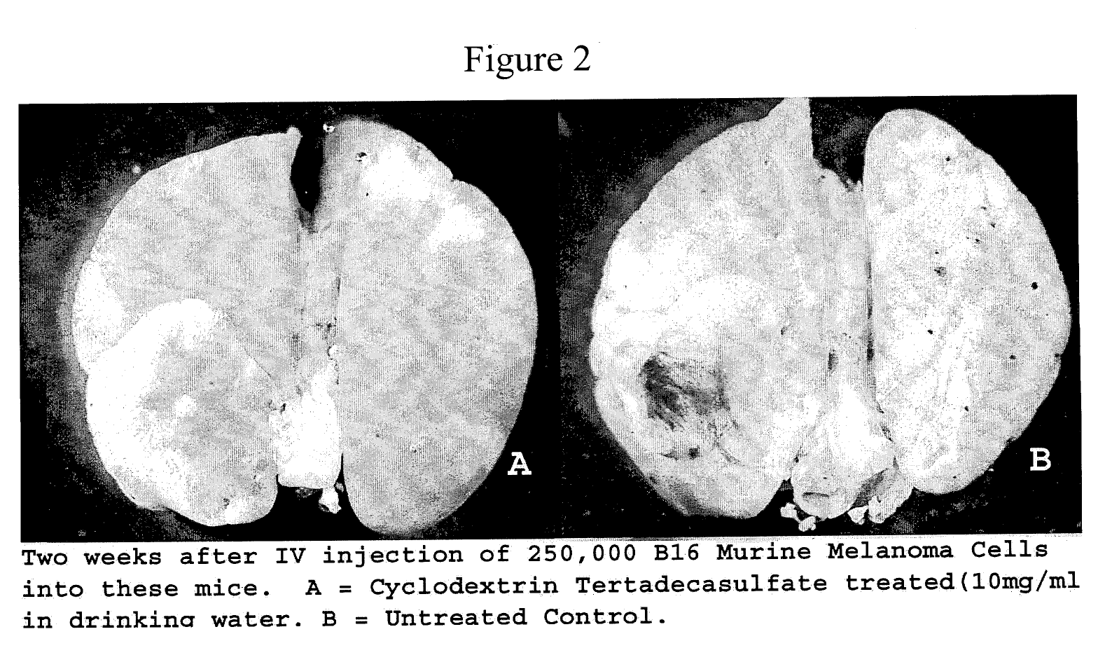

C57B1-6 mice (n=2) were treated with 10 mg/ml β-cyclodextrin tetradecasulfate in their drinking water for two weeks. The control animal (n=2) was given cyclodextrin-free drinking water. Treated animals (FIG. 2A) had ⅓ fewer lung metastasis when compared to the control animal (FIG. 2B).

What is claimed:

1. A pharmaceutical composition comprising a highly sulfated oligosaccharide and a pharmaceutically suitable excipient.

2. The pharmaceutical composition of claim 1, wherein said highly sulfated oligosaccharide is a sulfated cyclodextrin.

3. The pharmaceutical composition of claim 2, wherein said sulfated cyclodextrin is selected from the group consisting of α, β or γ cyclodextrin.

4. The pharmaceutical composition of claim 3, wherein said highly sulfated cyclodextrin is a β-cyclodextrin tetradecasulfate.

5. The pharmaceutical composition of claim 2, wherein said sulfated cyclodextrin is complexed with a therapeutic agent.

6. The pharmaceutical composition of claim 5, wherein said therapeutic agent is selected from the group consisting of an antitumor drug, antineoplastic drug, cytokine and anti-angiogenesis agent.

7. A method for slowing tumor growth, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient.

8. The method for slowing tumor growth of claim 7, wherein said highly sulfated oligosaccharide is a sulfated cyclodextrin.

9. The method for slowing tumor growth of claim 8, wherein said sulfated cyclodextrin is selected from the group consisting of α, β or γ cyclodextrin.

10. The method of claim 9, wherein said highly sulfated cyclodextrin is β-cyclodextrin tetradecasulfate.

11. The method of claim 8, wherein said sulfated cyclodextrin is complexed to a therapeutic agent.

12. The method of claim 11, wherein said therapeutic agent is selected from the group consisting of an antitumor drug, antineoplastic drug, cytokine and anti-angiogenesis agent.

13. A method for decreasing cell proliferation, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient.

14. The method for decreasing cell proliferation of claim 13, wherein said highly sulfated oligosaccharide is a sulfated cyclodextrin.

15. The method for decreasing cell proliferation of claim 14, wherein said sulfated cyclodextrin is selected from the group consisting of α, β or γ cyclodextrin.

16. The method of claim 15, wherein said highly sulfated cyclodextrin is β-cyclodextrin tetradecasulfate.

17. The method of claim 14, wherein said sulfated cyclodextrin is complexed to a therapeutic agent.

18. The method of claim 17, wherein said therapeutic agent is selected from the group consisting of an antitumor drug, antineoplastic drug, cytokine and anti-angiogenesis agent.

19. The method of claim 13, wherein said cell is a melanoma cell.

20. A method for decreasing neovascularization, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient.

21. The method for decreasing neovascularization of claim 20, wherein said highly sulfated oligosaccharide is a sulfated cyclodextrin.

22. The method for decreasing neovascularization of claim 21, wherein said sulfated cyclodextrin is selected from the group consisting of α, β or γ cyclodextrin.

23. The method of claim 22, wherein said highly sulfated cyclodextrin is β-cyclodextrin tetradecasulfate.

24. The method of claim 21, wherein said sulfated cyclodextrin is complexed to a therapeutic agent.

25. The method of claim 24, wherein said therapeutic agent is selected from the group consisting of an antitumor drug, antineoplastic drug, cytokine and anti-angiogenesis agent.

26. A kit for slowing tumor growth comprising (i) a first container that comprises a highly sulfated oligosaccharide and (ii) instructions for use.

27. The kit of claim 26, wherein said highly sulfated oligosaccharide is a sulfated cyclodextrin.

28. The kit of claim 27, wherein said sulfated cyclodextrin is selected from the group consisting of α, β or γ cyclodextrin.

29. The kit of claim 28, wherein said highly sulfated cyclodextrin is β-cyclodextrin tetradecasulfate.

30. The kit of claim 27, wherein said sulfated cyclodextrin is complexed to a therapeutic agent.

31. The kit of claim 30, wherein said therapeutic agent is selected from the group consisting of an antitumor drug, antineoplastic drug, cytokine and anti-angiogenesis agent.

32. A kit for decreasing cell proliferation comprising (i) a first container that comprises a highly sulfated oligosaccharide and (ii) instructions for use.

33. The kit of claim 32, wherein said highly sulfated oligosaccharide is a sulfated cyclodextrin.

34. The kit of claim 33, wherein said sulfated cyclodextrin is selected from the group consisting of α, β or γ cyclodextrin.

35. The kit of claim 34, wherein said highly sulfated cyclodextrin is β-cyclodextrin tetradecasulfate.

36. The kit of claim 33, wherein said sulfated cyclodextrin is complexed to a therapeutic agent.

37. The kit of claim 36, wherein said therapeutic agent is selected from the group consisting of an antitumor drug, antineoplastic drug, cytokine and anti-angiogenesis agent.

38. A kit for decreasing neovascularization comprising (i) a first container that comprises a highly sulfated oligosaccharide and (ii) instructions for use.

39. The kit of claim 38, wherein said highly sulfated oligosaccharide is a sulfated cyclodextrin.

40. The kit of claim 39, wherein said sulfated cyclodextrin is selected from the group consisting of α, β or γ cyclodextrin.

41. The kit of claim 40, wherein said highly sulfated cyclodextrin is β-cyclodextrin tetradecasulfate.

42. The kit of claim 39, wherein said sulfated cyclodextrin is complexed to a therapeutic agent.

43. The kit of claim 42, wherein said therapeutic agent is selected from the group consisting of an antitumor drug, antineoplastic drug, cytokine and anti-angiogenesis agent.

44. A method for blocking metastatic spread, comprising (i) providing a composition that comprises a highly sulfated oligosaccharide and a pharmaceutically suitable excipient and (ii) administering to a subject an effective amount of a highly sulfated oligosaccharide and a pharmaceutically suitable excipient.

45. The method for blocking metastatic spread of claim 44, wherein said highly sulfated oligosaccharide is a sulfated cyclodextrin.

46. The method for blocking metastatic spread of claim 45, wherein said sulfated cyclodextrin is selected from the group consisting of α, β or γ cyclodextrin.

47. The method of claim 46, wherein said highly sulfated cyclodextrin is β-cyclodextrin tetradecasulfate.

48. The method of claim 45, wherein said sulfated cyclodextrin is complexed to a therapeutic agent.

49. The method of claim 48, wherein said therapeutic agent is selected from the group consisting of an antitumor drug, antineoplastic drug, cytokine and anti-angiogenesis agent.
